(19) United States
(12) Patent Application Publication
(10) Pub. No.: US 2004/0242915 A1
(43) Pub. Date: Dec. 2, 2004

(54) Modified alumina catalyst

(76) Inventors: Sumitra Srinath, Karnataka (IN); Madhavan Sridharan, Karnataka (IN); Ganesh Sambasivam, Karnataka (IN)

Correspondence Address: Choate Hall & Stewart, Patent Group, Exchange Place, 53 State Street, Boston, MA, 02109, US

(21) Appl. No.: 10/490,249

(22) PCT Filed: Sep. 20, 2001
(86) PCT No.: PCT/IN01/00159

(51) Int. Cl. C07C 271/02

(52) U.S. Cl. 560/157; 502/341

(57) ABSTRACT

In one aspect, the invention provides a solid catalyst comprising an active alumina catalyst impregnated with a metal hydroxide. In another aspect, the invention provides a process for preparing the inventive alumina catalyst. In certain embodiments, the catalyst may be used for the protection of amines, alcohols and thiols with a wide variety of protecting agents. This procedure is widely applicable for N-protection of amino acids which finds wide industrial applications. The catalyst is also useful for carrying out nucleophilic substitutions of aromatic halides containing an electron-withdrawing group. A wide variety of nucleophiles chosen from amines, primary and secondary, aromatic and aliphatic, as well as alcohols and thiols have been successfully employed. The methodology involves simple techniques and easy work up procedures and is thus useful for large-scale industrial preparations. Additionally, the reactions avoid the use of harmful solvents and thus satisfy the need for green chemistry.

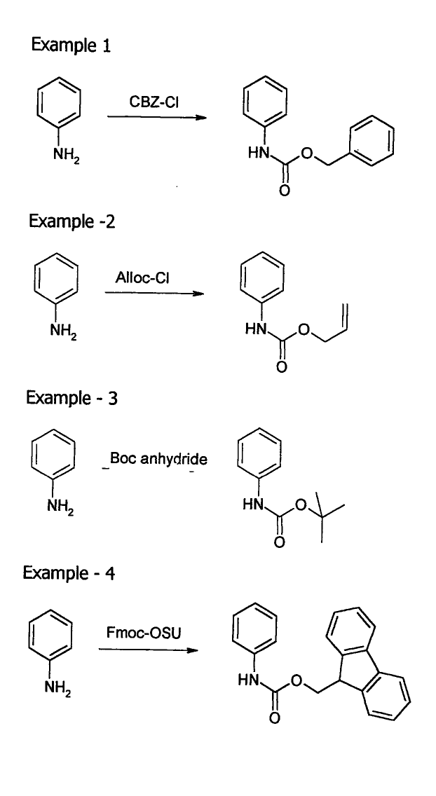

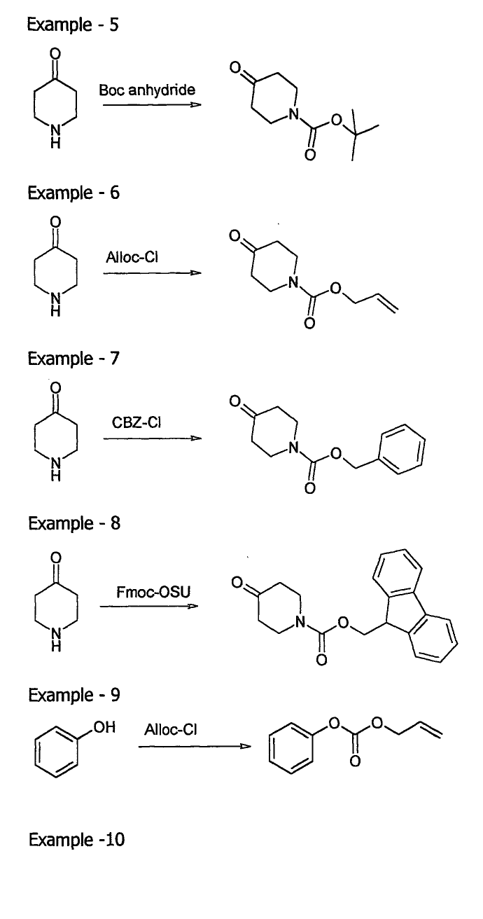

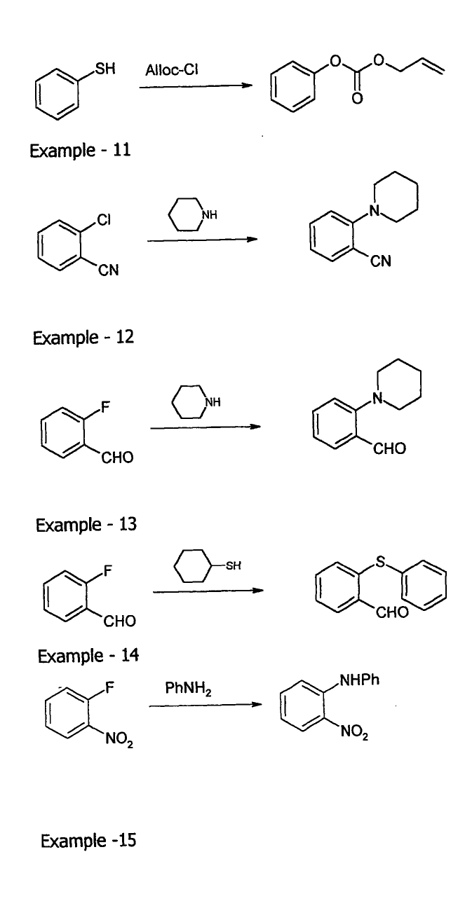

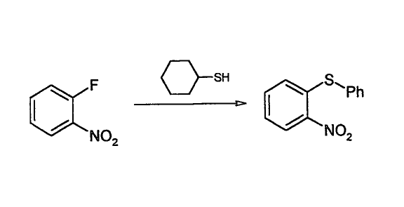

### PRIORITY INFORMATION

The present application claims the benefit under 35 U.S.C. § 371 of International Application No.: PCT/IN01/00591, filed Sep. 20, 2001, the entire contents of which is hereby incorporated herein by reference.

### FIELD OF THE INVENTION

In one aspect, the invention relates to an active alumina catalyst impregnated with a metal hydroxide chosen from alkali or alkaline earth metal, and a process for its preparation. The inventive catalyst may be used in protection reactions, in a wide variety of chemical transformations and for the nucleophilic substitution of aromatic halides containing an electron withdrawing group.

### BACKGROUND

Protection of compounds containing an active hydrogen (e.g., amine, phenols and thiols) forms an integral part of synthesis. Conventional protecting groups include t-BOC (di-tert-butyl dicarbonate), which reacts with amines to form a carbamate. Introduction of this BOC protection group, as described in the art, is typically carried out by reacting the amine with BOC anhydride in the presence of a base. Other amine protecting groups typically used include FMOC-Chloride for introducing a fluoromenthyl group, Alloc-Chloride for introducing an allyl carbamate group and CBZ-chloride for introducing a benzyloxy carbamate group.

Traditionally different reaction conditions are employed to introduce specific protective groups; for example, a basic reaction medium for introducing BOC and Alloc groups and an acidic medium for introducing the FMOC groups.

Aromatic nucleophilic substitution may be carried out by displacement of a halo group using strong nucleophiles. Reaction conditions are typically harsh however, and yields are often very poor. Improved methods have been developed for the displacement using a metal based catalyst, such as copper, and a strong base, such as potassium tert-butoxide. However, the reaction is specific and the yields are still poor. Mixture of palladium based reagents such as, palladium acetate, palladium hydroxide or palladium dibenzylidiene acetone complex, and a phosphine ligand in the presence of base, such as sodium tert-butoxide, offers a versatile procedure for the nucleophilic displacement of an aromatic halo group. However, the reaction utilizes phosphines, which are not readily available, and palladium reagents, which are expensive and possess disposal problems and are therefore not suitable for industrial scale operations.

A variety of methods are available in the literature for the nucleophilic substitution of aromatic halides. Palladium is the metal catalyst of choice for effecting the coupling of aromatic halides with a variety of nucleophiles. Reaction conditions often involve the use of trialkyl or triaryl phosphine and a palladium catalyst. Stephen Buchwald et al., (J. Org. Chem., 2000, 65, 1158-1174 and references cited therein), herein incorporated by reference, describe an efficient method for the amination of aryl chlorides, bromides and triflates using aryl phosphines, palladium complex and a base.

Scott Sawyer et al., (J. Org Chem, 1998, 63, 6338-6343), herein incorporated by reference, have demonstrated the synthesis of diaryl ethers, diaryl thioethers and diaryl amines by nucleophilic substitution of the aryl halides using KF-alumina, 18-crown-6 conditions. The disadvantage of this method, however, is that it utilizes 18-crown-6 as a complexing agent for the reaction. The reaction also requires a more tedious work-up procedure, which involves partitioning the reaction mixture between an organic solvent and aqueous media.

Furthermore, conventional aromatic nucleophilic substitution methods often use K₂CO₃-DMF. These conditions typically utilize high reaction temperatures that may be not readily employed in industrial scale operations.

### SUMMARY OF THE INVENTION

In one aspect, the invention provides a solid catalyst comprising an active alumina catalyst impregnated with a metal hydroxide. In certain embodiments, the metal hydroxide is an alkali or alkaline earth metal hydroxide.

In another aspect, the invention provides a process for preparing an alumina catalyst impregnated with a metal hydroxide, comprising steps of:

treating an aqueous solution of a metal hydroxide with alumina in an organic solvent; and

drying the resulting catalyst mixture.

In certain embodiments, the inventive catalyst may be useful for the introduction of protecting groups on amine, alcohol and/or thiol moieties. In certain other embodiments, the catalyst of the invention may be used to facilitate aromatic nucleophilic substitution.

In yet other embodiments, the catalyst of the present invention is easily removable from the reaction mixture thereby facilitating synthetic work-up procedures for the incorporation of protecting groups (e.g., for amines, alcohols and/or thiols) or for aromatic nucleophilic substitution reactions. In further embodiments, the inventive catalyst offers advantages, such as easy handling and disposal procedures of spent catalysts; environmentally friendly means to avoid the use of toxic and expensive solvents; and simple reaction conditions employable for industrial scale preparations.

In certain embodiments, the catalyst of the invention shows remarkable activity in reactions aimed at introducing protecting groups for amines, alcohols, phenols and thiols. In fact, a wide variety of protecting groups may be introduced in a mild and efficient manner at ambient temperatures using the catalyst of the invention.

In certain other embodiments, the inventive catalyst allows the use of reaction conditions, which can readily be scaled up for industrial scale preparations.

Other advantages to aspects and embodiments of the present invention include: easy preparation, and storage without appreciable activity loss, of the active catalyst; simple filtration to separate the active catalyst from the reaction products due to heterogeneousness of the catalyst; use of any solvent for the reaction (e.g. polar and/or non-polar); large volume handling capability due to the non-corrosiveness of the catalyst; and/or easy and environmentally friendly disposal methods and reaction conditions.

### DETAILED DESCRIPTION OF THE INVENTION

In one aspect, the invention provides a solid catalyst comprising an active alumina catalyst impregnated with a metal hydroxide. In certain embodiments, the metal hydroxide is an alkali or alkaline earth metal hydroxide.

In certain embodiments, the catalyst comprises an alumina impregnated with a base selected from an alkali or alkaline earth metal hydroxide. In certain exemplary embodiments, the metal hydroxide has the formula I:

M(OH)ₙ

wherein n is 1 or 2, and M is Li, Mg, Ca or Na.

In certain other exemplary embodiments, the metal hydroxide is lithium hydroxide. In certain exemplary embodiments, the lithium hydroxide content in alumina ranges from about 0.3 to about 3% by weight.

In another aspect, the invention provides a process for preparing alumina impregnated with lithium hydroxide. In certain embodiments, the resulting solid catalyst may be used for incorporating a wide variety of protecting groups on amines, alcohols, phenols and thiols. The preparation of fine chemicals, which are often used as starting materials for the preparation of active pharmaceuticals and/or intermediates thereof, frequently involves the protection of amine, alcohol, phenol and/or thiol groups.

In addition, N-protected amino acids and other amines, ethers and thioethers are often used as intermediates in a number of organic syntheses and/or preparations. Homogenous and heterogeneous catalyzed processes for the protection of these groups are known in the art.

In another aspect, the invention provides a process for preparing a catalyst containing alumina impregnated with a metal hydroxide, comprising: treating an aqueous solution of the metal hydroxide with alumina in an organic solvent; and drying the resulting catalyst mixture.

In certain embodiments, the step of drying is carried out at a temperature less than approximately 150° C.

In certain other embodiments, the organic solvent is dichloromethane, dioxane, toluene, acetonitrile or dimethyl formamide (DMF).

In yet other embodiments, the step of drying is carried out in vacuum.

In another aspect, the present invention provides a process for introducing protection groups using the alumina catalyst of the invention.

In certain embodiments, the catalyst is used for protecting primary and/or secondary amines. In certain exemplary embodiments, the primary and secondary amine may be an aromatic, aliphatic, heterocyclic, or cyclic amine. In yet other embodiments, the amine protecting group may be di-tert-butyl dicarbonate (Boc anhydride), 9-Fluorenylmethoxycarbonyl chloride (Fmoc-Cl), 9-Fluorenylmethoxy carbonyl N-hydroxy succinimide (Fmoc-OSu), Allyoxycarbonyl (Alloc), benzyl chloroformate (CBZ-Cl), acetic anhydride, trifluoroacetic anhydride, acid chloride, or sulfonyl chlorides.

In further embodiments, the catalyst is used for protecting primary, secondary and/or tertiary alcohols. In certain exemplary embodiments, the primary, secondary and tertiary alcohol may be an aromatic, aliphatic, heterocyclic, or cyclic alcohol. In yet other embodiments, the alcohol protecting group may be di-tert-butyl dicarbonate (Boc anhydride), 9-Fluorenylmethoxycarbonyl chloride (Fmoc-Cl), 9-Fluorenylmethoxy carbonyl N-hydroxy succinimide (Fmoc-OSu), Allyoxycarbonyl (Alloc), benzyl chloroformate (CBZ-Cl), acetic anhydride, trifluoroacetic anhydride, acid chloride, and sulfonyl.

In another embodiment, the catalyst is used for protecting primary, secondary and/or tertiary thiols. In certain exemplary embodiments, the primary, secondary and tertiary thiol may be an aromatic, aliphatic, heterocyclic, or cyclic thiol. In yet other embodiments, the thiol protecting group may be di-tert-butyl dicarbonate (Boc anhydride), 9-Fluorenylmethoxycarbonyl chloride (Fmoc-Cl), 9-Fluorenylmethoxy carbonyl N-hydroxy succinimide (Fmoc-OSu), Allyoxycarbonyl (Alloc), benzyl chloroformate (CBZ-Cl), acetic anhydride, trifluoroacetic anhydride, acid chloride, and sulfonyl chlorides.

In another aspect, the present invention provides a process for effecting aromatic nucleophilic substitution, comprising treating an aromatic halide with an amine, alcohol or thiol in the presence of the inventive alumina catalyst.

In certain embodiments of this aspect, the process comprises treating an aromatic halide with an amine in the presence of the inventive alumina catalyst to form the corresponding substituted aniline derivative. In certain exemplary embodiments, amine is a primary, or secondary amine. In certain other exemplary embodiments, the amine is an aromatic, aliphatic, heterocyclic or cyclic amine. In another embodiment, the aromatic halide contains an electron withdrawing group such as nitro, aldehyde, acid, ester, amide or nitrile.

In certain other embodiments of this aspect, the process comprises treating an aromatic halide with an alcohol in the presence of the inventive alumina catalyst to form the corresponding substituted ether derivative. In certain exemplary embodiments, the alcohol is a primary, secondary, or tertiary alcohol. In certain other exemplary embodiments, the alcohol is an aromatic, aliphatic, heterocyclic, or cyclic alcohol. In another embodiment, the aromatic halide contains an electron withdrawing group such as nitro, aldehyde, acid, ester, amide or nitrile.

In yet another embodiment, the process comprises treating an aromatic halide with a thiol in the presence of the inventive alumina catalyst to form the corresponding substituted thioether derivative. In certain exemplary embodiments, the thiol is a primary, secondary, or tertiary thiol. In certain other exemplary embodiments, the thiol is an aromatic, aliphatic, heterocyclic, or cyclic thiol. In a further embodiment, the aromatic halide contains an electron withdrawing group such as nitro, aldehyde, acid, ester, amide or nitrile.

As discussed above, in one aspect, the present invention provides a process for preparing an active alumina catalyst impregnated with a metal hydroxide. In certain embodiments, the inventive catalyst is a solid alumina-metal hydroxide catalyst. In certain embodiments, the metal hydroxide is an alkali or alkaline metal of general formula I:

M(OH)ₙ

wherein n is 1 or 2, and

M is Li Mg, Ca or Na.

In certain embodiments, the invention provides a process for protecting an amine group, comprising reacting an amine of formula II,

R₁R₂NH,

wherein R₁ and R₂ are independently H, alkyl, cycloalkyl, aryl, aralkyl, heteroalkyl, or heterocyclic; with the proviso that R₁ and R₂ are not both H.

with a protecting group such as di-tert-butyl dicarbonate (referred to as Boc anhydride), 9-Fluorenylmethoxycarbonyl chloride (Fmoc-Cl), 9-Fluorenylmethoxy carbonyl N-hydroxy succinimide (Fmoc-OSu), Allyoxycarbonyl (Alloc), benzyl chloroformate (CBZ-Cl), acetic anhydride, trifluoroacetic anhydride, acid chloride, or sulfonyl chlorides;

in the presence of a solid alumina-metal hydroxide catalyst of the invention.

In certain embodiments, the invention provides a process for protecting an alcohol group, comprising reacting an alcohol of formula III,

R₃OH,

wherein R₃ is alkyl, cycloalkyl, aryl, aralkyl, heterocyclic, heteroalkyl, or substituted aryl,

with a protecting group such as di-tert-butyl dicarbonate (referred to as Boc anhydride), 9-Fluorenylmethoxycarbonyl chloride (Fmoc-Cl), 9-Fluorenylmethoxy carbonyl N-hydroxy succinimide (Fmoc-OSu), Allyoxycarbonyl (Alloc), benzyl chloroformate (CBZ-Cl), acetic anhydride, trifluoroacetic anhydride, acid chloride, or sulfonyl chlorides;

in the presence of a solid alumina-metal hydroxide catalyst of the invention.

In certain embodiments, the invention provides a process for protecting a thiol group, comprising reacting a thiol of formula IV,

R₄SH,

wherein R₄ is alkyl, cycloalkyl, aryl, aralkyl, heterocyclic, heteroalkyl, or substituted aryl,

with a protecting group such as di-tert-butyl dicarbonate (referred to as Boc anhydride), 9-Fluorenylmethoxycarbonyl chloride (Fmoc-Cl), 9-Fluorenylmethoxy carbonyl N-hydroxy succinimide (Fmoc-OSu), Allyoxycarbonyl (Alloc), benzyl chloroformate (CBZ-Cl), acetic anhydride, trifluoroacetic anhydride, acid chloride, or sulfonyl chlorides;

in the presence of a solid alumina-metal hydroxide catalyst of the invention.

In certain other embodiments, the process resulting in desired protected compounds comprises reacting a substrate (e.g. amines, alcohols or thiols mentioned above) with a suitable protecting group in the presence of an active alumina catalyst containing a metal hydroxide, in a solvent such as dichloromethane, dioxane, toluene, acetonitrile, dimethyl formamide, dimethyl sulfoxide, diisopropyl ether, methyl tert-butyl ether, or cyclohexane, at room temperature; removing the active metal catalyst by filtration; and removing the solvent.

In certain embodiments, the invention provides a process for preparing an N-substituted aniline derivative, comprising reacting an aromatic halide of formula V,

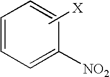

wherein X may be fluoro, chloro, or bromo and may be in ortho, meta or para position, with an amine of formula II,

R₁R₂NH

wherein R₁ and R₂ are independently H, alkyl, cycloalkyl, aryl, aralkyl, heteroalkyl, or heterocyclic; with the proviso that R₁ and R₂ are not both H,

in the presence of a solid alumina-metal hydroxide catalyst of the invention.

In certain embodiments, the invention provides a process for preparing an aromatic ether derivative, comprising reacting an aromatic halide of formula V,

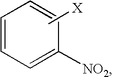

wherein X may be fluoro, chloro, or bromo and may be in ortho, meta or para position, with an alcohol of formula III,

R₃OH,

wherein R₃ is alkyl, cycloalkyl, aryl, aralkyl, heterocyclic, heteroalkyl, or substituted aryl,

in the presence of a solid alumina-metal hydroxide catalyst of the invention.

In certain embodiments, R₃ is phenyl or substituted phenyl.

In certain embodiments, the invention provides a process for preparing an aromatic thioether derivative, comprising reacting an aromatic halide of formula V,

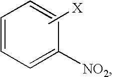

wherein X may be fluoro, chloro, or bromo and may be in ortho, meta or para position,

with a thiol of formula IV,

R₄SH,

wherein R₄ is alkyl, cycloalkyl, aryl, aralkyl, heterocyclic, heteroalkyl, or substituted aryl,

in the presence of a solid alumina-metal hydroxide catalyst of the invention.

In certain embodiments, the invention provides a process for preparing an N-substituted aniline derivative, comprising reacting an aromatic halide of formula VI,

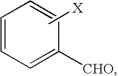

wherein X may be fluoro, chloro, or bromo and may be in ortho, meta or para position,

with an amine of formula II,

R₁R₂NH

wherein R₁ and R₂ are independently H, alkyl, cycloalkyl, aryl, aralkyl, heteroalkyl, or heterocyclic; with the proviso that R₁ and R₂ are not both H,

in the presence of a solid alumina-metal hydroxide catalyst of the invention.

In certain embodiments, the invention provides a process for preparing an aromatic ether derivative, comprising reacting an aromatic halide of formula VI,

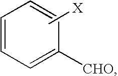

wherein X may be fluoro, chloro, or bromo and may be in ortho, meta or para position,

with an alcohol of formula III,

R₃OH,

wherein R₃ is alkyl, cycloalkyl, aryl, aralkyl, heterocyclic, heteroalkyl, or substituted aryl,

in the presence of a solid alumina-metal hydroxide catalyst of the invention.

In certain embodiments, R₃ is phenyl or substituted phenyl.

In certain embodiments, the invention provides a process for preparing an aromatic thioether derivative, comprising reacting an aromatic halide of formula VI,

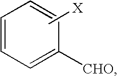

wherein X may be fluoro, chloro, or bromo and may be in ortho, meta or para position,

with a thiol of formula IV,

R₄SH,

wherein R₄ is alkyl, cycloalkyl, aryl, aralkyl, heterocyclic, heteroalkyl, or substituted aryl,

in the presence of a solid alumina-metal hydroxide catalyst of the invention.

In certain embodiments, the invention provides a process for preparing an N-substituted aniline derivative, comprising reacting an aromatic halide of formula VII,

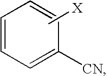

wherein X may be fluoro, chloro, or bromo and may be in ortho, meta or para position,

with an amine of formula II,

R₁R₂NH

wherein R₁ and R₂ are independently H, alkyl, cycloalkyl, aryl, aralkyl, heteroalkyl, or heterocyclic; with the proviso that R₁ and R₂ are not both H,

in the presence of a solid alumina-metal hydroxide catalyst of the invention.

The process described generally above was successfully used in the synthesis of 2-piperidinobenzonitrile (See, Example-11) which is an important intermediate in the synthesis of substituted phenyl acetamide, repaglinide which is used in the lowering of blood-sugar levels.

In certain embodiments, the invention provides a process for preparing an aromatic ether derivative, comprising reacting an aromatic halide of formula VII,

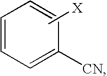

wherein X may be fluoro, chloro, or bromo and may be in ortho, meta or para position,

with an alcohol of formula III,

R₃OH,

wherein R₃ is alkyl, cycloalkyl, aryl, aralkyl, heterocyclic, heteroalkyl, or substituted aryl,

in the presence of a solid alumina-metal hydroxide catalyst of the invention.

In certain embodiments, R₃ is phenyl or substituted phenyl.

In certain embodiments, the invention provides a process for preparing an aromatic thioether derivative, comprising reacting an aromatic halide of formula VII,

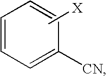

wherein X may be fluoro, chloro, or bromo and may be in ortho, meta or para position,

with a thiol of formula IV,

R₄SH,

wherein R₄ is alkyl, cycloalkyl, aryl, aralkyl, heterocyclic, heteroalkyl, or substituted aryl,

in the presence of a solid alumina-metal hydroxide catalyst of the invention.

In certain embodiments, the invention provides a process for preparing an N-substituted aniline derivative, comprising reacting an aromatic halide of formula VIII,

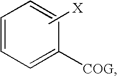

wherein X is F, Cl or Br;

G is OH, OR^{x} or NR^{y}R^{z};

R^{x}, R^{y} and R^{z} are independently alkyl, cycloalkyl, aryl, aralkyl, heterocyclic, heteroalkyl or substituted aryl; and

X may be in ortho, meta or para position,

with an amine of formula II,

R₁R₂NH

wherein R₁ and R₂ are independently H, alkyl, cycloalkyl, aryl, aralkyl, heteroalkyl, or heterocyclic; with the proviso that R₁ and R₂ are not both H,

in the presence of a solid alumina-metal hydroxide catalyst of the invention.

In certain embodiments, the invention provides a process for preparing an aromatic ether derivative, comprising reacting an aromatic halide of formula VIII,

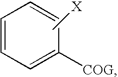

wherein X is F, Cl or Br;

G is OH, OR^{x} or NR^{y}R^{z};

R^{x}, R^{y} and R^{z} are independently alkyl, cycloalkyl, aryl, aralkyl, heterocyclic, heteroalkyl or substituted aryl; and

X may be in ortho, meta or para position,

with an alcohol of formula III,

R₃OH,

wherein R₃ is alkyl, cycloalkyl, aryl, aralkyl, heterocyclic, heteroalkyl, or substituted aryl,

in the presence of a solid alumina-metal hydroxide catalyst of the invention.

In certain embodiments, R₃ is phenyl or substituted phenyl.

In certain embodiments, the invention provides a process ifor preparing an aromatic thioether derivative, comprising reacting an aromatic halide of formula VIII,

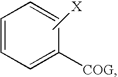

wherein X is F, Cl or Br;

G is OH, OR^{x} or NR^{y}R^{z};

R^{x}, R^{y} and R^{z} are independently alkyl, cycloalkyl, aryl, aralkyl, heterocyclic, heteroalkyl or substituted aryl; and

X may be in ortho, meta or para position,

with a thiol of formula IV,

R₄SH,

wherein R₄ is alkyl, cycloalkyl, aryl, aralkyl, heterocyclic, heteroalkyl, or substituted aryl,

in the presence of a solid alumina-metal hydroxide catalyst of the invention.

In certain other embodiments, the process resulting in desired aromatic nucleophilic substitution adducts comprises reacting a substrate (e.g. amines, alcohols or thiols) with an aromatic halide in the presence of an active alumina catalyst containing a metal hydroxide, in a solvent such as dichloromethane, dioxane, toluene, acetonitrile, dimethyl formamide, dimethyl sulfoxide, diisopropyl ether, methyl tert-butyl ether, or cyclohexane, room temperature; removing the active metal catalyst by filtration; and removing the solvent.

In certain embodiments, the reaction of amines (for example, aniline with aryl halides like 2-chloronitrobenzene) in dioxane afforded the corresponding N-substituted nitro anilines. The reaction appears to be quite general as aromatic nucleophilic substitution reactions using a variety of amines (e.g., primary and secondary aromatic, aliphatic, cycloalkyl amines, etc.), were attempted and proceeded fairly smoothly to give the corresponding N-substituted anilines.

The illustrated embodiments have been set forth only for the purposes of example and should not be taken as limiting the invention. Therefore, it should be understood that within the scope of the appended claims, the invention may be practiced other than specifically described herein.

### EXEMPLIFICATION

### Example 1

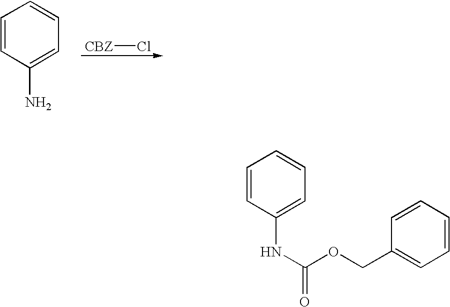

To a solution of 5 g of aniline in 50 ml of dichloromethane was added basic alumina impregnated with lithium hydroxide prepared by absorbing a 3N solution of lithium hydroxide (1.3. g LiOH) on 7.5 g of basic alumina (1.3 g of lithium hydroxide in 7.5 g of basic alumina). The resulting mixture was stirred for 5 minutes. Benzyl chloroformate (9.1 g) was slowly added over a period of 10 minutes, at room temperature. After 3 hours, the catalyst was filtered off and the solid bed was thoroughly washed with dichloromethane. The solvent was removed by evaporation, and the residue was crystallized from petroleum ether to give the desired product in 95% yield.

### Example 2

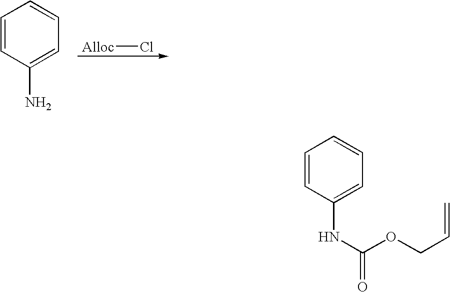

To a solution of 5 g of aniline in 50 ml of dichloromethane was added basic alumina impregnated with lithium hydroxide prepared by absorbing a 3N solution of lithium hydroxide (1.3. g LiOH) on 7.5 g of basic alumina (1.3 g of lithium hydroxide in 7.5 g of basic alumina). The resulting mixture was stirred for 5 minutes. Allyl chloroformate was slowly added over a period of 10 minutes, at room temperature. After 3 hours, the catalyst was filtered off and the solid bed was thoroughly washed with dichloromethane. The solvent was removed by evaporation, and the residue was crystallized from petroleum ether to give the desired product in 75% yield.

### Example 3

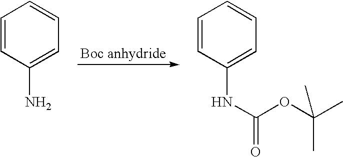

To a solution of 5 g of aniline in 50 ml of dichloromethane was added basic alumina impregnated with lithium hydroxide prepared by absorbing a 3N solution of lithium hydroxide (1.3. g LiOH) on 7.5 g of basic alumina (1.3 g of lithium hydroxide in 7.5 g of basic alumina). The resulting mixture was stirred for 5 minutes. Boc anhydride was slowly added over a period of 10 minutes, at room temperature. After 12 hours, the catalyst was filtered off and the solid bed was thoroughly washed with dichloromethane. The solvent was removed by evaporation, and the residue was crystallized from petroleum ether to give the desired product in 80% yield.

### Example 4

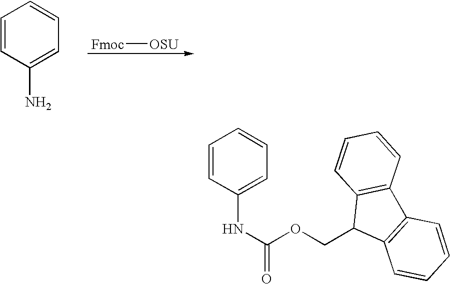

To a solution of 5 g of aniline in 50 ml of dichloromethane was added basic alumina impregnated with lithium hydroxide prepared by absorbing a 3N solution of lithium hydroxide (1.3. g LiOH) on 7.5 g of basic alumina (1.3 g of lithium hydroxide in 7.5 g of is basic alumina). The resulting mixture was stirred for 5 minutes. Fmoc-OSU was slowly added over a period of 10 minutes, at room temperature. After 12 hours, the catalyst was filtered off and the solid bed was thoroughly washed with dichloromethane. The solvent was removed by evaporation, and the residue was crystallized from petroleum ether to give the desired product in 95% yield.

### Example 5

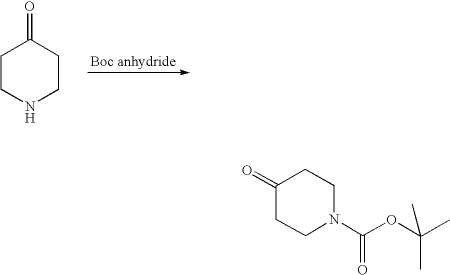

To a solution of 5 g of 4-piperidone in 50 ml of dichloromethane was added basic alumina impregnated with lithium hydroxide prepared by absorbing a 3N solution of lithium hydroxide (1.3. g LiOH) on 7.5 g of basic alumina (1.3 g of lithium hydroxide in 7.5 g of basic alumina). The resulting mixture was stirred for 5 minutes. Boc anhydride was slowly added over a period of 10 minutes, at room temperature. After 12 hours, the catalyst was filtered off and the solid bed was thoroughly washed with dichloromethane. The solvent was removed by evaporation, and the residue was crystallized from petroleum ether to give the desired product in 80% yield.

### Example 6

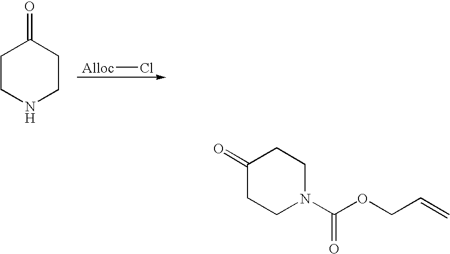

To a solution of 5 g of 4-piperidone in 50 ml of dichloromethane was added basic alumina impregnated with lithium hydroxide prepared by absorbing a 3N solution of lithium hydroxide (1.3. g LiOH) on 7.5 g of basic alumina (1.3 g of lithium hydroxide in 7.5 g of basic alumina). The resulting mixture was stirred for 5 minutes. Allyl chloroformate was slowly added to the resulting mixture over a period of 10 minutes, at room temperature. After 12 hours, the catalyst was filtered off and the solid bed was thoroughly washed with dichloromethane. The solvent was removed by evaporation, and the residue was crystallized from petroleum ether to give the desired product in 95% yield.

### Example 7

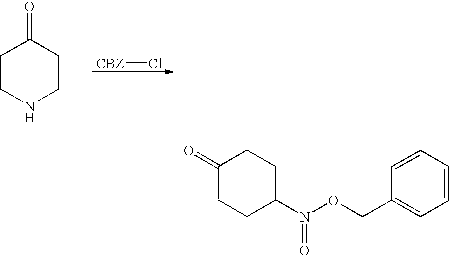

To a solution of 5 g of 4-piperidone in 50 ml of dichloromethane was added basic alumina impregnated with lithium hydroxide prepared by absorbing a 3N solution of lithium hydroxide (1.3. g LiOH) on 7.5 g of basic alumina (1.3 g of lithium hydroxide in 7.5 g of basic alumina). The resulting mixture was stirred for 5 minutes. Benzyl chloroformate was slowly added to the resulting mixture over a period of 10 minutes, at room temperature. After 3 hours, the catalyst was filtered off and the solid bed was thoroughly washed with dichloromethane. The solvent was removed by evaporation, and the residue was crystallized from petroleum ether to give the desired product in 95% yield.

### Example 8

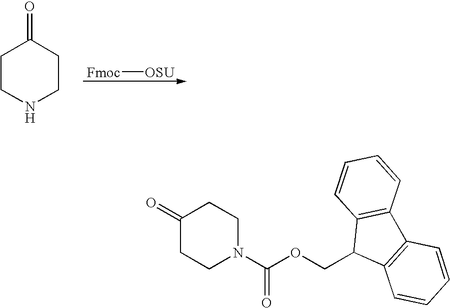

To a solution of 5 g of 4-piperidone in 50 ml of dichloromethane was added basic alumina impregnated with lithium hydroxide prepared by absorbing a 3N solution of lithium hydroxide (1.3. g LiOH) on 7.5 g of basic alumina (1.3 g of lithium hydroxide in 7.5 g of basic alumina). The resulting mixture was stirred for 5 minutes. Fmoc-OSU was slowly added over a period of 10 minutes, at room temperature. After 12 hours, the catalyst was filtered off and the solid bed was thoroughly washed with dichloromethane. The solvent was removed by evaporation, and the residue was crystallized from petroleum ether to give the desired product in 75% yield.

EXAMPLE 9

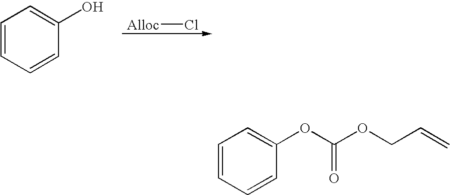

To a solution of 5 g of phenol in 50 ml of dichloromethane was added basic alumina impregnated with lithium hydroxide prepared by absorbing a 3N solution of lithium hydroxide (1.3. g LiOH) on 7.5 g of basic alumina (1.3 g of lithium hydroxide in 7.5 g of basic alumina). The resulting mixture was stirred for 5 minutes. Alloc-Cl was slowly added over a period of 10 minutes, at room temperature. After 12 hours, the catalyst was filtered off and the solid bed was thoroughly washed with dichloromethane. The solvent was removed by evaporation, and the residue was crystallized from petroleum ether to give the desired product in 85% yield.

### Example 10

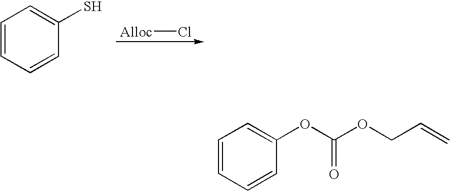

To a solution of 5 g of thiophenol in 50 ml of dichloromethane was added basic alumina impregnated with lithium hydroxide prepared by absorbing a 3N solution of lithium hydroxide (1.3. g LiOH) on 7.5 g of basic alumina (1.3 g of lithium hydroxide in 7.5 g of basic alumina). The resulting mixture was stirred for 5 minutes. Alloc-Cl was slowly added to the resulting mixture over a period of 10 minutes, at room temperature. After 12 hours, the catalyst was filtered off and the solid bed was thoroughly washed with dichloromethane. The solvent was removed by evaporation, and the residue was crystallized from petroleum ether to give the desired product in 70% yield.

### Example 11

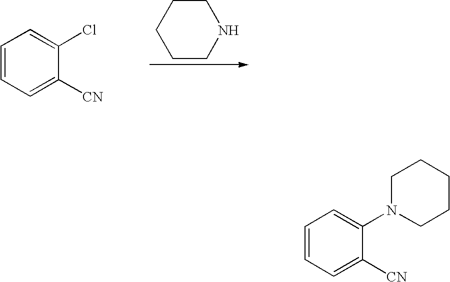

To a solution of 5 g of 2-chlorobenzonitrile in 50 ml of DMF was added basic alumina impregnated with lithium hydroxide prepared by absorbing a 3N solution of lithium hydroxide (2.7 g LiOH) on 7.5 g of basic alumina (2.7 g, 0.108M solution of lithium hydroxide in 7.5 g of basic alumina). The resulting mixture was stirred for 10 minutes. Piperidine was slowly added over a period of 10 minutes, and the resulting reaction mixture was refluxed at 120° C. Upon completion of the reaction, the reagent was filtered off and DMF was removed under reduced pressure. The residue was washed with water, extracted using ethyl acetate. Removal of ethyl acetate afforded the 2-(1-piperidinyl) benzonitrile in 50% yield.

### Example 12

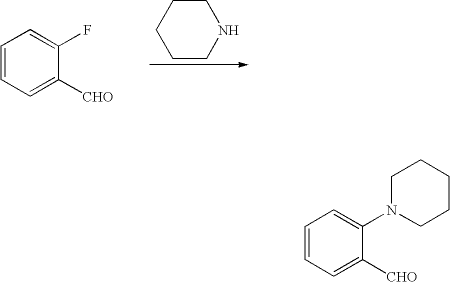

To a solution of 5 g of 2-fluorobenzaldehyde in 50 ml of DMF was added basic alumina impregnated with lithium hydroxide prepared by absorbing a 3N solution of lithium hydroxide (2.7 g LiOH) on 7.5 g of basic alumina (2.7 g, 0.108M solution of lithium hydroxide in 7.5 g of basic alumina). The resulting mixture was stirred for 10 minutes. Piperidine was slowly added over a period of 10 minutes, and the resulting reaction mixture was refluxed at 120° C. Upon completion of the reaction, the reagent was filtered off and DMF was removed under reduced pressure. The residue was washed with water, extracted using ethyl acetate. Removal of ethyl acetate afforded the 2-(1-piperidinyl) benzaldehyde in 80% yield.

### Example 13

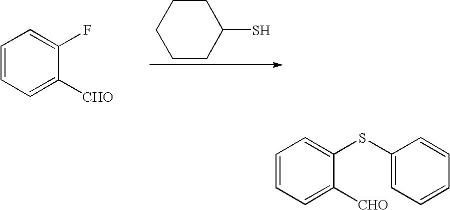

To a solution of 5 g of 2-fluorobenzaldehyde in 50 ml of DMF was added basic alumina impregnated with lithium hydroxide prepared by absorbing a 3N solution of lithium hydroxide (2.7 g LiOH) on 7.5 g of basic alumina (2.7 g, 0.108M solution of lithium hydroxide in 7.5 g of basic alumina). The resulting mixture was stirred for 10 minutes. Cyclohexanethiol was slowly added over a period of 10 minutes, and the resulting reaction mixture was refluxed at 120° C. Upon completion of the reaction, the reagent was filtered off and DMF was removed under reduced pressure. The residue was washed with water, extracted using ethyl acetate. Removal of ethyl acetate afforded the 2-(1-cyclohexylthio) benzaldehyde in 85% yield.

### Example 14

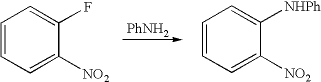

To a solution of 5 g of 2-fluoronitrobenzene in 50 ml of DMF was added basic alumina impregnated with lithium hydroxide prepared by absorbing a 3N solution of lithium hydroxide (2.7 g LiOH) on 7.5 g of basic alumina (2.7 g, 0.108M solution of lithium hydroxide in 7.5 g of basic alumina). The resulting mixture was stirred for 10 minutes. Aniline was slowly added over a period of 10 minutes, and the resulting reaction mixture was refluxed at 120° C. Upon completion of the reaction, the reagent was filtered off and DMF was removed under reduced pressure. The residue was washed with water, extracted using ethyl acetate. Removal of ethyl acetate afforded the 2-(N-phenylamino) nitrobenzene in 90% yield.

### Example 15

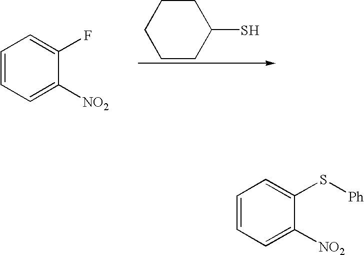

To a solution of 5 g of 2-fluoronitrobenzene in 50 ml of DMF was added basic alumina impregnated with lithium hydroxide prepared by absorbing a 3N solution of lithium hydroxide (2.7 g LiOH) on 7.5 g of basic alumina (2.7 g, 0.108M solution of lithium hydroxide in 7.5 g of basic alumina). The resulting mixture was stirred for 10 minutes. Cyclohexanethiol was slowly added over a period of 10 minutes, and the resulting reaction mixture was refluxed at 120° C. Upon completion of the reaction, the reagent was filtered off and DMF was removed under reduced pressure. The residue was washed s with water, extracted using ethyl acetate. Removal of ethyl acetate afforded the 2-(cyclohexylthio) nitrobenzene in 90% yield.

1. A catalyst comprising alumina impregnated with a metal hydroxide.

2. The catalyst of claim 16, wherein the alkali or alkaline earth hydroxide is of formula I: {M(OH)ₙ} wherein n is 1 or 2, and M is Li, Mg, Ca or Na.

3. The catalyst of claim 2, wherein the alkali or alkaline earth hydroxide is lithium hydroxide.

4. The catalyst of claim 3, wherein the lithium hydroxide is present in an amount ranging from about 0.3 to about 3% by weight.

5. A process for a catalyst containing alumina impregnated with a metal hydroxide, comprising:
treating an aqueous solution of the metal hydroxide with alumina in an organic solvent, and
drying the resulting catalyst mixture.

6. The process of claim 5, wherein the organic solvent is dichloromethane, dioxane, toluene, acetonitrile or dimethyl formamide(DMF).

7. The process of claim 5 or 6, wherein the step of drying is carried out in vacuum.

8. A process for protecting an amine group comprising treating a an amine of formula II:
R₁R₂NH,
wherein R₁ and R₂ are independently H, alkyl, cycloalky, aryl, aralkyl, heteroalkyl, or heterocyclic; with the proviso that R₁ and R₂ are not both H, a protecting group such as di-tert-butyl dicarbonate (Boc anhydride), 9-Fluorenylmethoxycarbonyl chloride (Fmoc-Cl), 9-Fluorenylmethoxy carbonyl N-hydroxy succinimide (Fmoc-OSu), Allyoxycarbonyl (Alloc), benzyl chloroformate (CBZ-Cl), acetic anhydride, trifluoroacetic anhydride, acid chloride, or sulfonyl chlorides in the presence of the catalyst of claim 1.

9. A process for protecting an alcohol group comprising treating a an alcohol of formula III:
R₃OH,
wherein R₃ is alkyl, cycloalkyl, aryl, aralkyl, heterocyclic, heteroalkyl, or substituted aryl, with a protecting group such as di-tert-butyl dicarbonate (Boc anhydride), 9-Fluorenylmethoxycarbonyl chloride (Fmoc-Cl), 9-Fluorenylmethoxy carbonyl N-hydroxy succinimide (Fmoc-OSu), Allyoxycarbonyl (Alloc), benzyl chloroformate (CBZ-Cl), acetic anhydride, trifluoroacetic anhydride, acid chloride, or sulfonyl chlorides;
in the presence of the catalyst of claim 1.

10. A process for protecting a thiol group comprising treating a a thiol of formula IV:
R₄SH,
wherein R₄ is alkyl, cycloalkyl, aryl, aralkyl, heterocyclic, heteroalkyl, or substituted aryl, a protecting group such as di-tert-butyl dicarbonate (Boc anhydride), 9-Fluorenylmethoxycarbonyl chloride (Fmoc-Cl), 9-Fluorenylmethoxy carbonyl N-hydroxy succinimide (Fmoc-OSu), Allyoxycarbonyl (Alloc), benzyl chloroformate (CBZ-Cl), acetic anhydride, trifluoroacetic anhydride, acid chloride, or sulfonyl chlorides;
in the presence of the catalyst of claim 1.

11. A process for effecting aromatic nucleophilic substitution, comprising treating an aromatic halide with an amine, alcohol or thiol in the presence of the catalyst of claim 1.

12. The process of claim 11, wherein the amine is of formula II:
R₁R₂NH,
wherein R₁ and R₂ are independently H, alkyl, cycloalkyl, aryl, aralkyl, heteroalkyl, or heterocyclic; with the proviso that R₁, and R₂ are not both H.

13. The process of claim 11, wherein the alcohol is of formula III:
R₃OH,
wherein R₃ is alkyl, cycloalkyl, aryl, aralkyl, heterocyclic, heteroalkyl, or substituted aryl.

14. The process of claim 11, wherein the thiol is of formula IV:
R₄SH,
wherein R₄ is alkyl, cycloalkyl, aryl, aralkyl, heterocyclic, heteroalkyl, or substituted aryl.

15. The process of claim 11, wherein the aromatic halide is 2-chlorobenzonitrile; and the amine is piperidine.

16. The catalyst of claim 1, wherein the metal hydroxide is an alkali or alkaline earth metal hydroxide.

17. The process of claim 11, wherein the aromatic halide has one of the following structures: 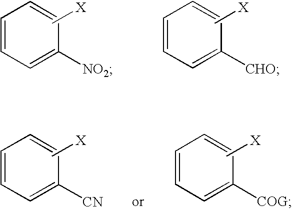
wherein X may be fluoro, chloro, or bromo and may be in ortho, meta or para position;
G is OH, OR^{x} or NR^{y}R^{z}; and
R^{x}, R^{y} and R^{z} are independently alky, cycloalkyl, aryl, aralkyl, heterocyclic, heteroalkyl or substituted aryl.

18. The process of claim 11, wherein the catalyst is of formula I: {M(OH)ₙ} wherein n is 1 or 2, and M is Li, Mg, Ca or Na.

19. The process of claim 18, wherein the catalyst is LiOH.
